(19) United States
(12) Patent Application Publication
(10) Pub. No.: US 2009/0169618 A1
(43) Pub. Date: Jul. 2, 2009

(54) Zolpidem pharmaceutical compositions

(76) Inventors: Limor Ari-Pardo, Beit Halevi (IL); Sivan Antler, Modiin (IL)

Correspondence Address: KENYON & KENYON LLP, ONE BROADWAY, NEW YORK, NY, 10004, US

(21) Appl. No.: 12/317,834

(22) Filed: Dec. 29, 2008

Related U.S. Application Data
(60) Provisional application No. 61/009,280, filed on Dec. 26, 2007.

(51) Int. Cl. A61K 31/437; A61K 9/54

(52) U.S. Cl. 424/458; 514/300

(57) ABSTRACT

The present invention provides extended release pharmaceutical compositions comprising zolpidem or a salt thereof.

### CROSS-REFERENCE TO RELATED APPLICATION

This application claims the benefit of U.S. Provisional Application No. 61/009,280, filed Dec. 26, 2007, hereby incorporated by reference.

### FIELD OF THE INVENTION

The present invention relates to extended release pharmaceutical compositions comprising zolpidem or a salt thereof, and a process for preparing the same.

### BACKGROUND OF THE INVENTION

Zolpidem, as a hemitartrate salt, is currently approved for the short-term treatment of insomnia in the United States under the trademark of AMBIEN®. Zolpidem hemitartrate is classified as a non-benzodiazepine hypnotic of the imidazopyridine class.

Zolpidem hemitartrate (CAS Registry No. 99294-93-6) has the chemical name imidazo[1,2-a]pyridine-3-acetamide,N,N,6-trimethyl-2-(4-methylphenyl)-(2R,3R)-2,3-dihydroxy-butanedioate. Zolpidem is represented by the structural formula:

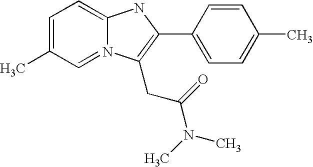

Zolpidem is apparently described in U.S. Pat. No. 4,382,938, and formulations thereof are understood to be disclosed in U.S. Pat. No. 6,514,531, both are incorporated herein by reference.

U.S. Pat. No. 6,514,531 would appear to relate to a zolpidem pharmaceutical controlled-release dosage form having a biphasic in-vitro dissolution profile, having an immediate release phase and a prolonged release phase, wherein 40 to 70% of the drug is released during the immediate release phase of 30 minutes, and wherein 90% of the total amount of zolpidem is released between 2 and 6 hours.

U.S. Pat. No. 6,638,535 would seem to describe a pellet comprised of zolpidem and the pellet-forming carrier microcrystalline cellulose. This pellet appears to exhibit a dissolution profile wherein 60% of the drug is released from the pellet within 5 minutes. This pellet does not necessarily require the presence of any rate-controlling agents. The release rate is reportedly controlled by the pellets' size, which is said to be within the range of 0.85 and 2 mm.

U.S. publication no. 2006/0159744 appears to relate to a zolpidem formulation having an in-vitro dissolution profile wherein 40 to 70% of the drug is released during an immediate release pulse of maximum 30 minutes, followed by a delayed release pulse of between 2 and 6 hours, during which 85% of the total amount of the drug is released. The delay of the second pulse is fixed, and it is between 50 and 200 minutes.

There is a need for additional pharmaceutical compositions of zolpidem, having a controlled dissolution rate. Further, there is a need to provide additional pharmaceutical compositions of zolpidem that are easy to manufacture, in which the release profile can be easily adjusted.

### SUMMARY OF THE INVENTION

In a first embodiment, the present invention provides a pharmaceutical composition comprising a plurality of pellets, wherein not more than 35% of the zolpidem or salt thereof present in the pharmaceutical composition dissolve in 30 minutes, as measured in vitro using a paddle at 50 rpm in 500 mL of dissolution medium composed of 0.01M HCl pH=2 or 0.01M phosphate buffer at 37° C.

In a second embodiment, the present invention provides a pharmaceutical composition comprising a plurality of pellets, wherein each pellet comprises: (1) a core comprising an admixture of zolpidem or a pharmaceutically acceptable salt thereof with at least one pharmaceutically acceptable excipient, and (2) an extended release layer disposed on the core. The extended release layer preferably covers the core, and the core is preferably monolithic.

In a third embodiment, the present invention provides a pharmaceutical composition comprising a mixture of:
1) a plurality of pellets coated with an extended release coating, and thus provide an extended release of the drug; and
2) a plurality of pellets wherein the cores are not coated with an extended release layer disposed thereon, and which therefore provide an immediate release of the drug,

wherein each pellet comprises a core comprising an admixture of zolpidem or a pharmaceutically acceptable salt thereof with at least one pharmaceutically acceptable excipient, and wherein the pellets of both populations are coated with a top coat layer.

The pharmaceutical compositions of any embodiment of the present invention preferably have a dissolution profile such that not more than 35% of the zolpidem or salt thereof present in the pharmaceutical composition dissolves within 30 minutes, as measured in vitro using a paddle at 50 rpm in 500 mL of dissolution medium composed of 0.01M HCl pH=2 or 0.01M phosphate buffer at 37° C.

In any embodiment of the present invention, the pharmaceutical composition preferably contains a pH modifier in the core.

In a further embodiment of the present invention, there is provided a process for preparing a pharmaceutical composition comprising:

(a) forming pellets comprising zolpidem or a pharmaceutically acceptable salt thereof with at least one pharmaceutically acceptable excipient; and

(b) applying an extended release layer onto the pellets.

A further coating of excipients (a top coat layer) may be applied to the pellets.

The present invention further provides a process for preparing a pharmaceutical composition containing multi-particulate delivery system as described above comprising two populations of pellets, wherein the process comprises:

(a) forming pellets comprising zolpidem or a pharmaceutically acceptable salt thereof with at least one pharmaceutically acceptable excipient;

(b) dividing the pellets into two populations;

(c) applying an extended release layer onto one of the populations of pellets; and

(d) applying a top coat layer.

The pellets of the present invention may be further compressed into tablets or may be filled into capsules or sachets.

### BRIEF DESCRIPTION OF THE FIGURES

FIG. 1 illustrates the zolpidem tartrate dissolution profile of the sample prepared according to Example 1.

FIG. 2 illustrates the zolpidem tartrate dissolution profile of the sample prepared according to Example 2.

FIG. 3 illustrates the zolpidem tartrate dissolution profile of the sample prepared according to Example 3.

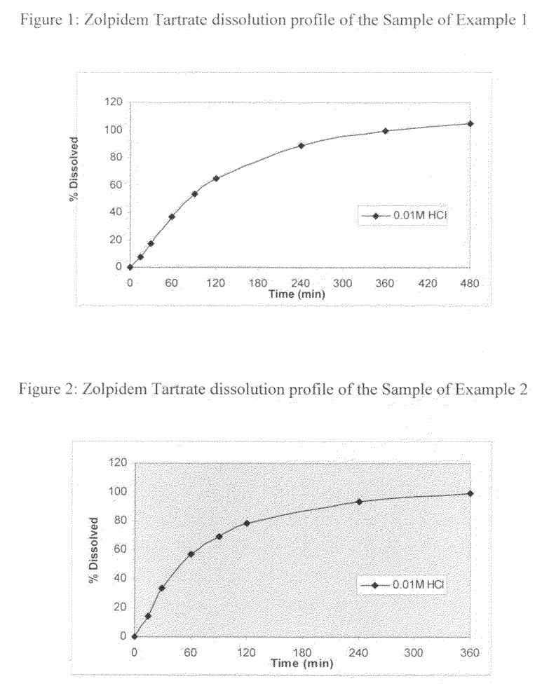

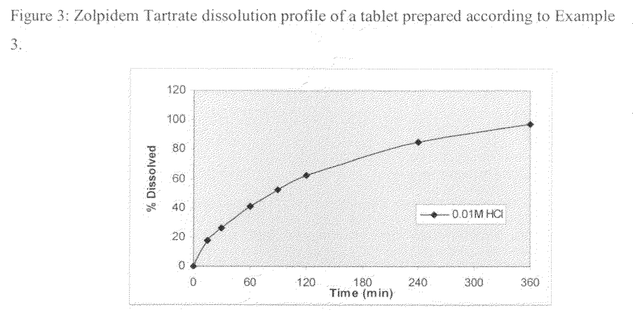

### DETAILED DESCRIPTION OF THE INVENTION

Unless otherwise indicated, dissolution profiles mentioned herein are measured using a dissolution apparatus according to US Pharmacopeia—i.e. using a paddle at 50 rpm in 500 mL of a dissolution medium of 0.01M HCl at pH 2 or 0.01 M phosphate buffer at 37° C.

The term “multi-particulate delivery system” in the present invention is interchangeable to the term “multi-particulate pharmaceutical composition”.

The term “immediate release” according to any embodiment of the present invention refers to a dissolution profile (as measured using the above dissolution apparatus) wherein at least about 25% by weight of the zolpidem or a pharmaceutically acceptable salt thereof is released from the population forming the immediate release pellets within 30 minutes. Preferably, the term “immediate release” refers to a dissolution profile wherein at least about 30% by weight of the zolpidem or a pharmaceutically acceptable salt thereof is released from the population forming the immediate release pellets within 30 minutes. Preferably, the term “immediate release” refers to a dissolution profile wherein at least about 50% by weight of the zolpidem or a pharmaceutically acceptable salt thereof is released from the population forming the immediate release pellets within 30 minutes. More preferably, the term “immediate release” refers to a dissolution profile wherein at least about 60% by weight of the zolpidem or a pharmaceutically acceptable salt thereof is released from the population forming the immediate release pellets within 30 minutes. Most preferably, the term “immediate release” refers to a dissolution profile wherein at least about 70% by weight of the zolpidem or a pharmaceutically acceptable salt thereof is released from the population forming the immediate release pellets within 30 minutes. Most preferably, the term “immediate release” refers to a dissolution profile wherein at least about 80% by weight of the zolpidem or a pharmaceutically acceptable salt thereof is released from the population forming the immediate release pellets within 30 minutes.

The term “extended release” refers to a dissolution profile wherein not more than about 35% by weight of the zolpidem or pharmaceutically acceptable salt thereof is released from the composition within 30 minutes. Preferably, the term “extended release” refers to a dissolution profile wherein not more than about 25% by weight of the zolpidem or pharmaceutically acceptable salt thereof is released from the composition within 30 minutes. More preferably, the term “extended release” refers to a dissolution profile wherein not more than about 20% by weight of the zolpidem or pharmaceutically acceptable salt thereof is released from the composition within 30 minutes.

As used herein, the term “lactose” includes all forms of lactose, with lactose monohydrate being preferred.

As used herein, polyethylene glycol (PEG) refers to any type of polyethylene glycol, however low molecular weight PEG is preferred (examples of which include PEG 200, PEG 300, PEG 400, PEG 540, PEG 600. PEG 400 is preferred.

As used herein, microcrystalline cellulose can be of any type (e.g. Avicel PH 101, Avicel PH 102, Avicel PH 103, Avicel 105, Avicel 112, Avicel PH 200). Preferably, the microcrystalline cellulose is Avicel PH101.

The term “top coat layer” according to any embodiment of the present invention refers to a coating comprising a coating agent, selected from: povidone, hypromellose, hydroxypropyl cellulose, methyl cellulose and preferably hypromellose and from a anti tacking agent selected from: glyceryl monostearate, talc, silicon dioxide, titanium dioxide, polyethylene glycol, magnesium stearate and preferably talc, titanium dioxide and polyethylene glycol.

As used herein, the term “core” relates to the part of the composition, composed of a monolithic structure with the drug dispersed within it as well as at least one pharmaceutical excipient.

Unless otherwise indicated, all references herein to zolpidem are intended to relate to zolpidem or a pharmaceutically salt thereof or mixtures thereof. A particularly preferred pharmaceutically acceptable salt of zolpidem in the formulations and processes of the present invention is zolpidem hemitartrate/zolpidem tartrate.

These compositions when administered to normal healthy adult males and females between the ages of 18 and 55, at a dose of about 12.5 mg, exhibit any or all of: a Cmax of zolpidem between 135 and 546 ng/mL; an average Cmax of 295 ng/mL; an AUC₀₋ₜ value of between 358 and 2768 ng-hr/mL; and an average AUC₀₋ₜ of 1222 ng-hr/mL.

The NDA 021-774 for the product currently sold as AmbienCR® lists in the Approved Drug Products with Therapeutic Equivalence Evaluations (Orange Book) U.S. Pat. No. 6,514,531, and it is therefore understood that the Ambien CR® is an embodiment of the '531 invention.

Despite the fact that the dissolution rate of the compositions of the present invention is different than the one in the '531 patent, preferred embodiments of the present invention are bioequivalent to marketed embodiment of the '531 patent.

Preferably, the compositions of the present invention are pharmaceutical compositions in the form of a multi-particulate delivery system, more preferably comprising a plurality of pellets.

Pellets are understood to be generally rounded, if not entirely spherical, particles, which are typically the result of extrusion and spheronizing an active composition.

In a first embodiment, the present invention provides a pharmaceutical composition comprising a plurality of pellets, wherein not more than 35% of the zolpidem or salt thereof present in the pharmaceutical composition dissolve in 30 minutes, as measured in vitro using a paddle at 50 rpm in 500 mL of dissolution medium composed of 0.01M HCl pH=2 or 0.01M phosphate buffer at 37° C. Optionally the pellets may further be coated with a top coat layer.

In another embodiment, the present invention provides a pharmaceutical composition comprising a plurality of pellets, wherein each pellet comprises: (1) a core comprising an admixture of zolpidem or a pharmaceutically acceptable salt thereof with at least one pharmaceutically acceptable excipient, and (2) an extended release layer disposed on the core. The extended release layer preferably covers the core, and the core is preferably monolithic.

In another embodiment, the present invention provides a pharmaceutical composition comprising a mixture of:
1) a plurality of pellets coated with an extended release coating, and thus provide an extended release of the drug; and
2) a plurality of pellets wherein the cores are not coated with an extended release layer disposed thereon, and which therefore provide an immediate release of the drug,

wherein each pellet comprises a core comprising an admixture of zolpidem or a pharmaceutically acceptable salt thereof with at least one pharmaceutically acceptable excipient, and wherein the pellets of both populations are coated with a top coat layer.

The multiparticulate dosage forms of the present invention thus provide a mixture of immediate release and extended release dissolution profiles by providing populations which are coated with an extended release layer, and populations that are not coated with an extended release layer. The proportions of each population can be selected in order to achieve any desired dissolution profile. The advantage of such a multiparticulate delivery system is that it allows for an easy adjustment of drug release rate by simply varying the proportions of the populations that comprise it. Further, since the cores for each population of the pellets can be made by the same process, the process for manufacturing the multiparticulate delivery system is particularly easy—the cores can be divided into two populations afterwards, and the population forming the extended release pellets can simply be coated with the extended release coating, whereas the population forming the immediate release pellets are not subjected to coating with the extended release coating. The populations can be subsequently combined in suitable proportions to achieve any desired dissolution profile, and then compressed into tablets or filled into capsules or sachets. The present invention avoids the need to form multi-layer tablets in order to achieve immediate- and extended-release profiles in one dosage form. Further, the compositions and processes of the present invention avoid the need to apply the zolpidem as a coating, since the zolpidem or a pharmaceutically acceptable salt thereof is dispersed in the core of the pellet (i.e. to form a monolithic core structure).

Thus, the invention provides a pharmaceutical composition comprising a mixture of: (1) a plurality of pellets as described in any of the preceding embodiments, which are coated with an extended release coating, and thus provide an extended release of the drug, and (2) a plurality of pellets wherein each pellet comprises a core comprising an admixture of zolpidem or a pharmaceutically acceptable salt thereof with at least one pharmaceutically acceptable excipient, wherein the core is as defined in any of the preceding embodiments, wherein the cores are not coated with an extended release layer disposed thereon, and which therefore provide an immediate release of the drug, and wherein the pellets of both populations are coated with a top coat layer.

Preferably, the extended release pellets are about 70% by weight of the population of pellets.

Conveniently, the pharmaceutical compositions of the invention are in the form of a compressed dosage form such as a tablet, or alternatively, the pellets may be filled into capsules or sachets.

In any embodiment of the present invention, the zolpidem or a pharmaceutically acceptable salt thereof is preferably present in an amount of about 4% to about 10%, preferably about 5% to about 9%, and more preferably about 7% to about 8%, by weight of the core. Preferably the zolpidem is present in the form of zolpidem hemitartrate. The zolpidem or pharmaceutically acceptable salt thereof is preferably present in an amount of about 1% to about 10%, more preferably about 2% to about 3.5% by weight relative to the weight of the pharmaceutical composition (e.g. tablet).

The pharmaceutical compositions of any embodiment of the present invention preferably have a dissolution profile such that not more than about 35% of the zolpidem or salt thereof present in the pharmaceutical composition dissolves within 30 minutes, as measured in vitro using a paddle at 50 rpm in 500 mL of dissolution medium composed of 0.01M HCl pH=2 or 0.01M phosphate buffer at 37° C.

In any embodiment of the present invention, the pharmaceutical composition preferably contains a pH modifier in the core. In a preferred embodiment of all aspects of the invention described herein, the pH modifier employed in the compositions of the present invention is an organic acid or a salt thereof. Examples of useful pH modifiers include a pharmaceutically acceptable organic acids or salts thereof, preferably selected from the group consisting of malic acid, tartaric acid, citric acid, fumaric acid, lactic acid, maleic acid and succinic acid, glutaric acid, glutamic acid and mandelic acid. Preferably, the pH modifier is tartaric acid, malic acid, fumaric acid, lactic acid, citric acid, succinic acid or a salt thereof. More preferably the pH modifier is selected from tartaric acid, citric acid or glutaric acid or a salt thereof, and is most preferably tartaric acid.

The pH modifier is included in the composition in order to enable release of zolpidem or a salt thereof in the intestine by lowering the micro environment pH within the composition.

In any embodiment of the present invention, the pH modifier is present in an amount of about 2% to about 20%, preferably about 5% to about 17%, and more preferably about 8% to about 14%, by weight relative to the weight of the core. Preferably the pH modifier is present in an amount of about 1% to about 5%, more preferably about 2% to about 4% by weight of the pharmaceutical composition (e.g. tablet). The pH modifier may be used in a weight ratio of pH modifier:zolpidem or a pharmaceutically acceptable salt thereof from about 3:1 to about 1:1, preferably about 2:1 to about 1:1, and more preferably about 1.6:1 to about 1.4:1, and particularly preferably about 1.5:1.

The cores which contain the active agent and form part of the pellets of the pharmaceutical compositions of the invention are preferably “monolithic”, i.e. the cores are formed as a single solid phase, which is essentially uniform or homogeneous. These are distinguished over cores such as those formed by coating the active agent onto, e.g. inert spheres.

In any embodiment of the pharmaceutical compositions of the present invention, the weight ratio of the total excipient in the core:zolpidem or a pharmaceutically acceptable salt thereof, is from about 18:1 to about 8:1, preferably about 16:1 to about 10:1, and more preferably about 14:1 to about 12:1.

In a preferred embodiment, the pharmaceutical compositions of the present invention preferably comprises a core which is comprised of an admixture of zolpidem with at least one pellet former, and at least one diluent.

The at least one pharmaceutically acceptable excipient in any embodiment of the present invention preferably includes at least one pellet former in the core. Particularly preferred pellet formers are those that can be used in extrusion-spheronization processes (the preferred method of manufacture of the pellets of the present invention). Examples of suitable pellet formers include those selected from the group consisting of microcrystalline cellulose, lactose monohydrate, sucrose, starch or mixtures thereof. Microcrystalline cellulose is the preferred pellet former excipient for use in the compositions of the present invention.

The pellet former (such as microcrystalline cellulose) is preferably present in an amount of about 30% to about 85%, preferably about 50% to about 75%, and more preferably about 60% to about 70%, by weight relative to the weight of the core. The pellet former can be present in an amount of about 15% to about 30%, preferably about 18% to about 25%, and more preferably about 20% to about 25% by weight relative to the weight of the pharmaceutical composition (e.g. tablet).

The pharmaceutical compositions of any embodiment of the present invention may also include a diluent in the core. Useful diluents include those selected from the group consisting of lactose, cellulose, mannitol, dextrin, dextrose, sorbitol, starch, sucrose, talc, tragacanth, xylitol, and mixtures thereof. Preferably, the diluent is selected from the group consisting of lactose, cellulose, mannitol, sorbitol, starch and talc, and mixtures thereof. Lactose, preferably in the form of lactose monohydrate, is a preferred diluent in any composition of the present invention.

In any embodiment of the present invention, the diluent may be present in an amount of about 5% to about 25%, preferably about 10% to about 20% and more preferably about 12% to about 16%, by weight relative to the weight of the core. In any embodiment of the present invention, the weight ratio of diluent:zolpidem or a pharmaceutically acceptable salt thereof (preferably zolpidem hemitartrate) is in the range of about 5:1 to about 1:1, preferably about 3:1 to about 1:1, and more preferably about 2:1.

whenever the cores are provided with an extended release layer, this layer is composed of materials that will act to extend the release of the drug from the composition. The extended release layer comprises at least one release modifying agent and optionally at least one plasticizer. The extended release layer preferably comprises at least one polymer, such as hydroxypropylcellulose, hydroxypropyl methylcellulose, ethylcellulose and polymethacrylates. The extended release layer is preferably a water insoluble polymer such as ethyl cellulose. In any embodiment of the present invention, the release modifying agent is preferably present in an amount of about 50% to about 90%, preferably about 70% to about 85%, and more preferably about 78% to about 82% by weight relative to the weight of the extended release layer. Typically, the release modifying agent can be present in an amount of about 1% to about 8%, preferably about 1.5% to about 6%, and most preferably about 2% to about 5.5% by weight relative to the weight of the pharmaceutical composition (e.g. tablet).

In any embodiment of the present invention, a plasticizer is preferably included in the extended release layer. The plasticizer can be hydrophilic such as triethyl citrate and polyethylene glycol and/or hydrophobic such as diethyl phthalate, dibutyl phthalate, dibutyl sebacate and acetyl tributyl citrate. It can be easily determined that other materials may be substituted for the polymer/plasticizer if they fulfill the same function i.e. they serve to control the release of the drug at a rate commensurate with the instant invention. It is possible that various hydrophobic materials including oils and waxes may be equally useful in this regard and can be found through routine experimentation or in the wealth of relevant literature. Preferably the plasticizer is selected from the group consisting of dibutyl sebacate, diethyl phthalate, glycerine, polyethylene glycol, propylene glycol, sorbitol, triacetin, and triethyl citrate, and mixtures thereof. Dibutyl sebacate, diethylphthalate, polyethylene glycol and propylene glycol, and mixtures thereof are particularly preferred. Most preferably, the plasticizer is a mixture of polyethylene glycol (preferably PEG 400) and dibutylsebacate.

The plasticizer is preferably present in an amount of about 5% to about 40%, preferably about 10% to about 30%, and more preferably about 15% to about 25%, by weight relative to the weight of the extended release layer. Typically, in preferred embodiments of the present invention, the weight ratio of release modifying agent to plasticizer in the extended release layer is from about 7:1 to about 2:1, preferably from about 6:1 to about 3:1, and more preferably about 5:1 to about 3:1, and most preferably about 4:1.

In any embodiment of the present invention, the extended release layer is preferably present in an amount of about 8% to about 20%, and preferably about 10% to about 15%, by weight relative to the combined weight of the core and the extended release layer.

In preferred embodiments of the compositions of the present invention, the pellets are further mixed with at least one pharmaceutically acceptable excipient, preferably selected from one or more of a filler, binder, glidant, disintegrant and lubricant. In a more preferred embodiment of any of the compositions of the present invention, the pellets are further mixed with at least one pharmaceutically acceptable excipient selected from the group consisting of a filler, glidant, disintegrant and lubricant. Preferably, the pellets are further mixed with a mixture of sprayed dried composition of lactose monohydrate and starch (preferably a spray-dried compound consisting of 85% alpha-lactose monohydrate and 15% maize starch, such as Starlac®), colloidal silicon dioxide, sodium starch glycolate and magnesium stearate.

Preferably, the pellets of any embodiment of the present invention may be optionally mixed with at least one pharmaceutically acceptable excipient, before being compressed into tablets (or filled into capsules or sachets). Most preferably, the pellets of both populations are mixed with a mixture of a filler, glidant, disintegrant and lubricant selected from the groups described above.

Suitable fillers are preferably selected from the group consisting of microcrystalline cellulose, lactose monohydrate, maize starch, powdered cellulose, sorbitol and mannitol and mixtures thereof. A particularly preferred filler is lactose monohydrate in the form of a spray-dried compound consisting of 85% alpha-lactose monohydrate and 15% maize starch (such as Starlac®). Suitable binders include those selected from the group consisting of povidone, hydroxypropyl cellulose, and hydroxypropyl methylcellulose and mixtures thereof. Suitable glidants are preferably selected from the group consisting of talc and silicon dioxide and mixtures thereof. Suitable disintegrants are preferably selected from the group consisting of croscarmellose sodium, pregelatinized starch, crospovidone, hydroxypropyl cellulose, and sodium starch glycolate and mixtures thereof. Suitable lubricants are preferably selected from the group consisting of stearic acid, magnesium stearate, mineral oil, hydrogenated castor oil and sodium stearyl fumarate.

The pellets as described in any of the embodiments of the present invention as described above can be incorporated into multiparticulate dosage forms, such as the ones described above.

In the preferred multiparticulate pharmaceutical compositions of the present invention, the populations (extended release and release populations) of pellets are admixed and the composition is in the form of a compressed dosage form or is filled into a capsule or a sachet. The preferred multiparticulate pharmaceutical compositions of the present invention are in the form of tablets. In another aspect, the present invention provides a process for preparing the compositions described above.

Thus, the invention further provides a process for preparing a pharmaceutical composition comprising:

(a) forming pellets comprising zolpidem or a pharmaceutically acceptable salt thereof with at least one pharmaceutically acceptable excipient; and

(b) applying an extended release layer onto the pellets.

A further coating of excipients (top coat layer) may be applied to the pellets. Preferably the top coat layer is selected from: a mixture of hypromellose with at least one of titanium dioxide, polyethylene glycol and talc.

The pellets are preferably formed by wet granulation of a mixture comprising zolpidem or a pharmaceutically acceptable salt thereof, a pellet former, optionally a pH modifier and at least one pharmaceutically acceptable excipient, preferably a diluent, followed by extrusion spheronization to form cores i.e. extrusion of the wet mass to form rod-shaped particles of uniform size and spheronization to round off the rods into spherical particles. The cores are then dried and the dried cores are optionally screened in order to achieve the desired particle size distribution. Suitable pellet former, diluents and pH modifiers are described above.

The cores may then be coated with an extended release layer containing at least one release modifier and at least one plasticizer. Suitable release modifiers and plasticizers are described above.

The coated pellets can be further mixed with a mixture of a filler, glidant, disintegrant and lubricant. Preferably the pellets are mixed with a mixture of a sprayed dried composition of lactose monohydrate and starch, colloidal silicon dioxide, sodium starch glycolate and magnesium stearate.

The process can further comprise compressing the pellets into tablets or filling the pellets into capsules or sachets.

The invention further provides a process for preparing a pharmaceutical composition containing the multiparticulate pharmaceutical compositions as described above comprising:

(a) forming pellets comprising zolpidem or a pharmaceutically acceptable salt thereof with at least one pharmaceutically acceptable excipient;

(b) dividing the pellets into two populations; and

(c) applying an extended release layer onto one of the populations of pellets; and

(d) applying a top coat layer.

The pellets may be formed as described above, and then coating the pellets with an extended release layer, preferably wherein the extended release layer contains at least one release modifier and at least one plasticizer. The release modifier and plasticizer are preferably as described above.

Each population of pellets, i.e. both extended release pellets and immediate release pellets are further coated with a top coating as described above.

The pellet populations can be admixed and then mixed with at least one pharmaceutically acceptable excipient, preferably selected from one or more of a filler, binder, glidant, disintegrant and lubricant as described above.

The pellet populations are then compressed into tablets or are filled into capsules or sachets.

In any aspect of the invention described herein, the extended release layer may be applied to the cores in any conventional method. Preferably, the extended release layer is applied using a solution of at least one polymer and at least one plasticizer. The coating process may be adequately performed using a fluidized bed coater, preferably equipped with a Wurster device (bottom spray). Other equipment that may be useful are well known in the art.

In any aspect of the invention described herein, the processes for preparing a composition of the invention preferably further comprises mixing a plurality of the particles (e.g. pellets), which provide an extended release of zolpidem or a salt thereof, with at least one pharmaceutically acceptable excipient such as, but not limited to, a filler, binder, glidant, disintegrant or lubricant. The mixture can be filled into capsules or sachets or compressed into tablets. It is understood that in the embodiment where a single coated particle is envisioned, it is most often a coated tablet itself.

Preferably the pharmaceutical compositions of the present invention are in the form of tablets, capsules or sachets, wherein the weight of the tablet, or the weight of the capsule/sachet filling is about 200 mg to about 800 mg, preferably about 300 mg to about 600 mg, more preferably about 450 mg to about 550 mg.

Particularly preferred pharmaceutical compositions according to the present invention comprise a pellet comprising (wt % are relative to the weight of the tablet or capsule/sachet filling): a core, wherein said core comprises:
microcrystalline cellulose, preferably in an amount of about 15-40 wt %, more preferably about 20-25 wt %,
tartaric acid, preferably in an amount of about 1-6 wt %, more preferably about 2-4 wt %
lactose monohydrate, preferably in an amount of about 2-7 wt %, more preferably about 4-5 wt %
zolpidem tartrate, preferably in an amount of about 0.5-5 wt %, more preferably about 2-3 wt %

and an extended release coating, wherein said extended release coating comprises:
ethyl cellulose, preferably in an amount of about 1-8 wt %, more preferably about 2-4 wt %
PEG 400, preferably in an amount of about 0.1-2 wt %, more preferably about 0.2-0.8 wt %
dibutyl sebacate, preferably in an amount of about 0.1-2 wt %, more preferably about 0.2-0.8 wt %.

The pellet may optionally be mixed with the following excipients before being compressed into tablets (or filled into sachets or capsules) (wt % are relative to the weight of the tablet or capsule/sachet filling):
a spray-dried compound consisting of 85% alpha-lactose monohydrate and 15% maize starch, such as Starlac®, preferably in an amount of about 30-70 wt %, more preferably in an amount of about 40-60 wt % and most preferably about 50-54 wt %
colloidal silicon dioxide, preferably in an amount of about 0.5-5 wt %, preferably about 0.8-3 wt % and most preferably about 1.2-1.6 wt %
sodium starch glycolate, preferably in an amount of about 1-10 wt %, more preferably about 2-8 wt %, and most preferably about 4-5.5 wt %
magnesium stearate, preferably in an amount of about 0.4-1.5 wt %, more preferably about 0.3-1.5 wt % and most preferably about 0.4-1.2 wt %.

The pellets can be further coated with a top coating selected from the following (wt % are relative to the weight of the tablet or capsule/sachet filling):
hypromellose, preferably in an amount of about 0.5-3 wt %, more preferably about 0.7-2 wt %
titanium dioxide, preferably in an amount of about 0.5-2 wt %, more preferably about 1-1.4 wt %

or
hypromellose, preferably in an amount of about 0.5-3 wt %, more preferably about 0.7-2 wt %
PEG 400, preferably in an amount of about 0.2-2 wt %, more preferably about 0.5-1.0 wt %

or
hypromellose, preferably in an amount of about 0.5-3 wt %, more preferably about 0.7-2 wt %
talc, preferably in an amount of about 1.0-3.0 wt %, more preferably about 1.4-1.7 wt %.

In the preferred multiparticulate dosage forms of the present invention, the composition comprises:

an extended release population comprising a core containing
microcrystalline cellulose, preferably in an amount of about 15-40 wt %, more preferably about 20-25 wt %
tartaric acid, preferably in an amount of about 1-6 wt %, more preferably about 2-4 wt %
lactose monohydrate, preferably in an amount of about 2-7 wt %, more preferably about 4-5 wt %
zolpidem tartrate, preferably in an amount of about 0.5-5 wt %, more preferably about 2-3 wt %

and an extended release coating comprising:
ethyl cellulose, preferably in an amount of about 1-8 wt %, more preferably about 2-4 wt %
PEG 400, preferably in an amount of about 0.1-2 wt %, more preferably about 0.2-0.8 wt %
dibutyl sebacate, preferably in an amount of about 0.1-2 wt %, more preferably about 0.2-0.8 wt %
   and an immediate release population comprising a core containing:
microcrystalline cellulose, preferably in an amount of about 15-40 wt %, more preferably about 20-25 wt %
tartaric acid, preferably in an amount of about 1-6 wt %, more preferably about 2-4 wt %
lactose monohydrate, preferably in an amount of about 2-7 wt %, more preferably about 4-5 wt %
zolpidem tartrate, preferably in an amount of about 0.5-5 wt %, more preferably about 2-3 wt %

In the multiparticulate compositions each of the immediate release and extended release population of pellets are further coated with a coating selected from the following (wt % are relative to the weight of the tablet or capsule/sachet filling):
hypromellose, preferably in an amount of about 0.5-3 wt %, more preferably about 0.7-2 wt %
titanium dioxide, preferably in an amount of about 0.5-2 wt %, more preferably about 1-1.4 wt %

or
hypromellose, preferably in an amount of about 0.5-3 wt %, more preferably about 0.7-2 wt %
PEG 400, preferably in an amount of about 0.2-2 wt %, more preferably about 0.5-1.0 wt %

or
hypromellose, preferably in an amount of about 0.5-3 wt %, more preferably about 0.7-2 wt %
talc, preferably in an amount of about 1.0-3.0 wt %, more preferably about 1.4-1.7 wt %.

The pellets of the multiparticulate dosage forms may optionally be mixed with the following excipients before being compressed into tablets (or filled into capsules or sachets) (wt % are relative to the weight of the tablet or capsule/sachet filling):
a spray-dried composition consisting of 85% alpha-lactose monohydrate and 15% maize starch, such as Starlac® in an amount of about 30-70 wt %, preferably 40-65 wt %, more preferably 50-60 wt %
colloidal silicon dioxide in an amount of about 0.5-3 wt %, preferably 0.8-2 wt %, more preferably 1.2-1.7 wt %
magnesium stearate in an amount of about 0.2-2 wt %, preferably 0.3-1.5 wt %, more preferably 0.4-1.2 wt %
and optionally sodium starch glycolate in an amount of about 1-10 wt %, preferably 2-6 wt %, more preferably 4-5.5 wt %

In any aspect of the invention described herein, the pellets comprising zolpidem preferably have an average diameter of from about 0.1 to about 1.0 micrometers, preferably about 0.2 to about 0.8 micrometers and more preferably about 0.3 to about 0.7 micrometers.

Having described the invention with reference to certain preferred embodiments (aspects), other embodiments will become apparent to one skilled in the art from consideration of the specification. The invention is further defined by reference to the following examples describing in detail the preparation of the compositions and methods for preparing thereof. It will be apparent to those skilled in the art that many modifications, both to materials and methods, may be practiced without departing from the scope of the invention.

### EXAMPLES

### Example 1

Granulated inner cores are prepared by combining microcrystalline cellulose (121.25 mg/tab), tartaric acid (18.75 mg/tab), lactose monohydrate (25.0 mg/tab), zolpidem tartrate (12.5 mg/tab) using extrusion-spheronization technology.

The granulated inner cores are coated with an extended release layer made up of ethyl cellulose (26.8 mg/tab), polyethylene glycol (3.35 mg/tab), diutyl sebacate 3.35 mg/tab). The extended release coated spheres are mixed with Starlac (264.5 mg/tab), colloidal silicon dioxide (7.5 mg/tab), sodium starch glycolate (25 mg/tab) and magnesium stearate (5 mg/tab) and compressed into round tablets.

### Example 2

Granulated inner cores are prepared by combining microcrystalline cellulose (111.25 mg/tab), tartaric acid (18.75 mg/tab), lactose monohydrate (25.0 mg/tab), zolpidem tartrate (12.5 mg/tab) using extrusion-spheronization technology.

The granulated inner cores are coated with an extended release layer made up of ethyl cellulose (18.8 mg/tab), polyethylene glycol (2.35 mg/tab), dibutyl sebacate (2.35 mg/tab).

The extended release coated spheres are mixed with starlac (253.5 mg/tab), colloidal silicon dioxide (7.0 mg/tab), sodium starch glycolate (24 mg/tab) and magnesium stearate (2.5 mg/tab) and compressed into round tablets.

### Example 3

Granulated inner cores are prepared by combining microcrystalline cellulose (113.5 mg/tablet), lactose monohydrate 200 mesh (25 mg/tablet), zolpidem tartrate (12.5 mg/tablet) and tartaric acid (19 mg/tablet) using extrusion-spheronization technology. The granulated inner cores are divided into two populations: for IR (immediate release) spheres and for ER (extended release) spheres. 70% by weight of the granulated inner cores are used to form the extended release population, and the remaining 30% by weight of the granulate inner cores are used to form the immediate release population. Each population is coated as follows:

The granulated inner cores for ER spheres are coated with an extended release layers made up of ethyl cellulose (11.2 mg/tablet), polyethylene glycol (1.4 mg/tablet) and dibutyl sebacate (1.4 mg/tablet). The extended release coated spheres are then coated with a top coat layer made up of hypromellose (4.0 mg/tablet) and titanium dioxide (6.0 mg/tablet).

The granulated inner cores for IR spheres are coated with a top coat layer made up of hypromellose (1.6 mg/tablet) and titanium dioxide (2.4 mg/tablet).

The top coated IR spheres and top coated ER spheres are mixed with StarLac® (260.5 mg/tablet), colloidal silicon dioxide (7.5 mg/tablet), sodium starch glycolate (25 mg/tablet) and magnesium stearate (5.0 mg/tablet) and then compressed into round tablets.

### Example 4

Granulated inner cores are prepared by combining microcrystalline cellulose (111.25 mg/tablet), lactose monohydrate 200 mesh (25 mg/tablet), zolpidem tartrate (12.5 mg/tablet) and tartaric acid (18.75 mg/tablet) using extrusion-spheronization technology. The granulated inner cores are divided into two populations: for IR (immediate release) spheres and for ER (extended release) spheres. 70% by weight of the granulated inner cores are used to form the extended release population, and the remaining 30% by weight of the granulate inner cores are used to form the immediate release population. Each population is coated as follows:

The granulated inner cores for ER spheres are coated with an extended release layers made up of ethyl cellulose (18.75 mg/tablet), polyethylene glycol (2.35 mg/tablet) and dibutyl sebacate (2.35 mg/tablet). The extended release coated spheres are then coated with a top coat layer made up of hypromellose (10.0 mg/tablet) and polyethylene glycol (4.0 mg/tablet).

The granulated inner cores for IR spheres are coated with a top coat layer made up of hypromellose (3.5 mg/tablet) and polyethylene glycol (1.5 mg/tablet).

The top coated IR spheres and top coated ER spheres are mixed with StarLac (275.65 mg/tablet), colloidal silicon dioxide (7.9 mg/tablet), sodium starch glycolate (24 mg/tablet) and magnesium stearate (5.25 mg/tablet) and then compressed into round tablets.

### Example 5

Granulated inner cores are prepared by combining microcrystalline cellulose (111.25 mg/tablet), lactose monohydrate 200 mesh (25 mg/tablet), zolpidem tartrate (12.5 mg/tablet) and tartaric acid (18.75 mg/tablet) using extrusion-spheronization technology. The granulated inner cores are divided into two populations: for IR (immediate release) spheres and for ER (extended release) spheres. 70% by weight of the granulated inner cores are used to form the extended release population, and the remaining 30% by weight of the granulate inner cores are used to form the immediate release population. Each population is coated as follows:

The granulated inner cores for ER spheres are coated with an extended release layers made up of ethyl cellulose (18.75 mg/tablet), polyethylene glycol (2.35 mg/tablet) and dibutyl sebacate (2.35 mg/tablet). The extended release coated spheres are then coated with a top coat layer made up of hypromellose (5.9 mg/tablet) and talc (8.4 mg/tablet).

The granulated inner cores for IR spheres are coated with a top coat layer made up of hypromellose (2.0 mg/tablet) and talc (3.0 mg/tablet).

The top coated IR spheres and top coated ER spheres are mixed with StarLac (305.22 mg/tablet), colloidal silicon dioxide (7.9 mg/tablet) and magnesium stearate (2.63 mg/tablet) and then compressed into round tablets.

### Bioequivalence Study

A relative bioequivalence study under fasting conditions of 12.5 mg Zolpidem extended-release tablets made according to Example 4 was held.

In this study, 12.5 mg Zolpidem Tartrate Extended-Release Tablets (lot # K-38150) manufactured by TEVA Pharmaceuticals Industries, Ltd. was compared with 12.5 mg AMBIEN CR™ Tablets (lot # 26524) distributed by Sanofi-Synthelabo Inc., USA; manufactured in France.

### Study Design

Randomized, single-dose, three-way crossover study under fasting conditions

Study Subjects
Eighteen healthy adult (male and female) subjects and no alternates
Female subjects practicing an acceptable method of birth control as judged by the investigator(s), such as condom with spermicide, condom and diaphragm, non-hormonal intrauterine device (IUD), or abstinence throughout the duration of the study; or of postmenopausal (no menses) status for at least 1 year, surgically sterile (bilateral tubal ligation, bilateral oophorectomy, or hysterectomy).
The subject is 18 years of age or older at the time of dosing.
The subject must have a body mass index (BMI) between 19-30 kg/m², inclusive, and weigh at least 110 lbs.

### Dosing Regimen

12.5 mg of test Zolpidem Tartrate Extended-Release Tablets (lot # K-38150, manufactured by TEVA Pharmaceuticals Industries, Ltd.) or 12.5 mg of reference Zolpidem Tartrate Extended-Release Tablets (AMBIEN CR™ Tablets distributed by Sanofi-Synthelabo Inc., USA; manufactured in France)
1 tablet of Test Product (A) with 8 fl. ozs. room temperature water after an overnight fast
1 tablet of Reference Product (C) with 8 fl. ozs. room temperature water after an overnight fast

### Drug Regimen

Three dosing periods

### Washout

At least 7 days

### Confinement

Approximately 10.5 hours prior to and until at least 24 hours after dosing each period.

TABLE 1
Summary of Cₘₐₓ and AUC₀₋ₜ of Test Product A (Lot # K-38150)

| Subject | Cₘₐₓ | AUC₀₋ₜ |
|---|---|---|
| 1 | 157 | 1064.15 |
| 2 | 307 | 967.23 |
| 3 | 268 | 1631.03 |
| 4 | 155 | 856.75 |
| 5 | 190 | 988.58 |
| 6 | 306 | 2380.67 |
| 7 | 253 | 1225.77 |
| 8 | 164 | 806.53 |
| 9 | 194 | 1120.10 |
| 10 | 233 | 1345.73 |
| 11 | 546 | 2768.52 |
| 12 | 151 | 358.27 |
| 13 | 264 | 1147.97 |
| 14 | 268 | 1019.42 |
| 15 | 135 | 554.84 |
| 16 | 189 | 775.32 |
| 17 | 469 | 1759.62 |
| 18 | 157 | 1064.15 |

TABLE 2
Bioequivalence studies comparing the invention's multi particulate
dosage form against a bi-layer tablet (prior art patent 531′)

| | Cmax | AUCₜ |
|---|---|---|
| Example 4 | 119.5% | 97.5% |

1. A pharmaceutical composition comprising a plurality of pellets containing zolpidem and at least one pharmaceutically acceptable excipient, wherein not more than 35% of the zolpidem or salt thereof present in the pharmaceutical composition dissolve in 30 minutes, as measured in vitro using a paddle at 50 rpm in 500 mL of dissolution medium composed of 0.01M HCl pH=2 or 0.01M phosphate buffer at 37° C.

2. A pharmaceutical composition comprising a plurality of pellets, wherein each pellet comprises: (1) a core comprising an admixture of zolpidem or a pharmaceutically acceptable salt thereof with at least one pharmaceutically acceptable excipient, and (2) an extended release layer disposed on the core.

3. A pharmaceutical composition comprising a mixture of:
(1) a plurality of pellets coated with an extended release coating, and thus provide an extended release of the drug; and
(2) a plurality of pellets wherein the cores are not coated with an extended release layer disposed thereon, and which therefore provide an immediate release of the drug,
wherein each pellet comprises a core comprising an admixture of zolpidem or a pharmaceutically acceptable salt thereof with at least one pharmaceutically acceptable excipient, and wherein the pellets of both populations are coated with a top coat layer.

4. The pharmaceutical composition according to any preceding claim in the form of a compressed dosage form, a capsule or a sachet.

5. The pharmaceutical composition according to any preceding claim in the form of a tablet.

6. The pharmaceutical composition according to any preceding claim wherein the zolpidem or a pharmaceutically acceptable salt thereof is present in an amount of about 4% to about 10%, preferably about 5% to about 9%, and more preferably about 7% to about 8%, by weight of the core.

7. The pharmaceutical composition according to any preceding claim wherein the zolpidem is present in the form of zolpidem hemitartrate.

7. The pharmaceutical composition according to claim 3, wherein the zolpidem or the pharmaceutically acceptable salt thereof is present in an amount of about 5% to about 9% by weight of the core.

8. The pharmaceutical composition according to claim 3, wherein the zolpidem or the pharmaceutically acceptable salt thereof is present in an amount of about 7% to about 8% by weight of the core.

9. The pharmaceutical composition according to claim 3, wherein the zolpidem is present in the form of zolpidem hemitartrate.

10. The pharmaceutical composition according to claim 1 or claim 2, wherein each pellet is coated with a top coat layer.

11. The pharmaceutical composition according to claim 3, wherein said top coat layer is selected from: a mixture of hypromellose with at least one of titanium dioxide, polyethylene glycol or talc.

12. The pharmaceutical compositions according to claims 2 or 3, wherein the dissolution profile is such that not more than about 35% of the zolpidem or salt thereof present in the pharmaceutical composition dissolves within 30 minutes, as measured in vitro using a paddle at 50 rpm in 500 mL of dissolution medium composed of 0.01M HCl pH=2 or 0.01M phosphate buffer at 37° C.

13. The pharmaceutical composition according to claim 3, wherein the at least one pharmaceutically acceptable excipient includes a pH modifier.

14. The pharmaceutical composition according to claim 13, wherein the pH modifier is a pharmaceutically acceptable acid.

15. The pharmaceutical composition according to claim 14, wherein the pharmaceutically acceptable organic acid is selected from the group consisting of malic acid, tartaric acid, citric acid, fumaric acid, lactic acid, maleic acid and succinic acid, glutaric acid, glutamic acid and mandelic acid.

16. The pharmaceutical composition according to claim 15, wherein the pH modifier is selected from tartaric acid, citric acid or glutaric acid.

17. The pharmaceutical composition according to claim 16, wherein the pH modifier is tartaric acid.

18. The pharmaceutical composition according claim 13, wherein the pH modifier is present in an amount of about 2% to about 20% by weight relative to the weight of the core.

19. The pharmaceutical composition according claim 18, wherein the pH modifier is present in an amount of about 5% to about 17% by weight relative to the weight of the core.

20. The pharmaceutical composition according claim 19, wherein the pH modifier is present in an amount of about 8% to about 14% by weight relative to the weight of the core.

21. The pharmaceutical composition according to claim 20, wherein the weight ratio of pH modifier to zolpidem or a pharmaceutically acceptable salt thereof is from about 3:1 to about 1:1.

22. The pharmaceutical composition according to claim 21, wherein the weight ratio of pH modifier to zolpidem or a pharmaceutically acceptable salt thereof is from about 2:1 to about 1:1.

23. The pharmaceutical composition according to claim 22, wherein the weight ratio of pH modifier to zolpidem or a pharmaceutically acceptable salt thereof is from about 1.6:1 to about 1.4:1.

24. The pharmaceutical composition according to claim 23, wherein the weight ratio of pH modifier to zolpidem or a pharmaceutically acceptable salt thereof is about 1.5:1.

25. The pharmaceutical composition according to claim 3, wherein the pharmaceutically acceptable excipient includes at least pellet former.

26. The pharmaceutical composition according to claim 25, wherein the pellet former is selected from the group consisting of microcrystalline cellulose, lactose monohydrate, sucrose, starch, and mixtures thereof.

27. The pharmaceutical composition according to claim 26, wherein the pellet former is microcrystalline cellulose.

28. The pharmaceutical composition according to claim 25, wherein the pellet former is present in an amount of about 30% to about 85% by weight relative to the weight of the core.

29. The pharmaceutical composition according to claim 28, wherein the pellet former is present in an amount of about 50% to about 75% by weight relative to the weight of the core.

30. The pharmaceutical composition according to claim 29, wherein the pellet former is present in an amount of about 60% to about 70% by weight relative to the weight of the core.

31. The pharmaceutical composition according to claim 3, wherein the pharmaceutically acceptable excipient includes a diluent.

32. The pharmaceutical composition according to claim 31, wherein the diluent is selected from the group consisting of lactose, cellulose, mannitol, dextrin, dextrose, sorbitol, starch, sucrose, talc, tragacanth, xylitol, and mixtures thereof.

33. The pharmaceutical composition according to claim 32, wherein the diluent is selected from the group consisting of lactose, cellulose, mannitol, sorbitol, starch, talc, and mixtures thereof.

34. The pharmaceutical composition according to claim 33, wherein the diluent is lactose.

35. The pharmaceutical composition according to claim 31, wherein the diluent is present in an amount of about 5% to about 25% by weight relative to the weight of the core.

36. The pharmaceutical composition according to claim 35, wherein the diluent is present in an amount of about 10% to about 20% by weight relative to the weight of the core.

37. The pharmaceutical composition according to claim 36, wherein the diluent is present in an amount of about 12% to about 16% by weight relative to the weight of the core.

38. The pharmaceutical composition according to claim 3, wherein the core comprises an admixture of zolpidem with at least one pellet former, and at least one diluent.

39. The pharmaceutical composition according to claim 38, wherein the pellet former is microcrystalline cellulose and the diluent is lactose.

40. The pharmaceutical composition according to claim 38, wherein the core further comprises a pH modifier and the pH modifier is tartaric acid.

41. The pharmaceutical composition according to claim 3, wherein the extended release layer comprises at least one release modifying agent and optionally at least one plasticizer.

42. The pharmaceutical composition according to claim 41, wherein the release modifying agent comprises at least one water insoluble polymer.

43. The pharmaceutical composition according to claim 42, wherein the release modifying agent is selected from the group consisting of ethylcellulose, hydroxypropylcellulose, hydroxypropyl methylcellulose and polymethacrylates.

44. The pharmaceutical composition according to claim 43, wherein the release modifying agent is ethylcellulose.

45. The pharmaceutical composition according to claim 41, wherein the release modifying agent is present in an amount of about 50% to about 90% by weight relative to the weight of the extended release layer.

46. The pharmaceutical composition according to claim 45, wherein the release modifying agent is present in an amount of about 70% to about 85% by weight relative to the weight of the extended release layer.

47. The pharmaceutical composition according to claim 46, wherein the release modifying agent is present in an amount of about 78% to about 82% by weight relative to the weight of the extended release layer.

48. The pharmaceutical composition according to claim 41, wherein the plasticizer is selected from the group consisting of dibutyl sebacate, diethyl phthalate, glycerine, polyethylene glycol, propylene glycol, sorbitol, triacetin, triethyl citrate, and mixtures thereof.

49. The pharmaceutical composition according to claim 48, wherein the plasticizer is a mixture of polyethylene glycol and dibutylsebacate.

50. The pharmaceutical composition according to claim 49, wherein the polyethylene glycol is PEG400.

51. The pharmaceutical composition according to claim 41, wherein the plasticizer is present in an amount of about 5% to about 40% by weight relative to the weight of the extended release layer.

52. The pharmaceutical composition according to claim 51, wherein the plasticizer is present in an amount of about 10% to about 30% by weight relative to the weight of the extended release layer.

53. The pharmaceutical composition according to claim 52, wherein the plasticizer is present in an amount of about 15% to about 25% by weight relative to the weight of the extended release layer.

54. The pharmaceutical composition according to claim 41, wherein the weight ratio of release modifying agent to plasticizer in the extended release layer is from about 7:1 to about 2:1.

55. The pharmaceutical composition according to claim 54, wherein the weight ratio of release modifying agent to plasticizer in the extended release layer is from about 6:1 to about 3:1.

56. The pharmaceutical composition according to claim 55, wherein the weight ratio of release modifying agent to plasticizer in the extended release layer is from about 5:1 to about 3:1.

57. The pharmaceutical composition according to claim 56, wherein the weight ratio of release modifying agent to plasticizer in the extended release layer is about 4:1.

58. The pharmaceutical composition according to claim 3, wherein the extended release layer is present in an amount of about 8% to about 20% by weight relative to the combined weight of the core and the extended release layer.

59. The pharmaceutical composition according to claim 58, wherein the extended release layer is present in an amount of about 10% to about 15% by weight relative to the combined weight of the core and the extended release layer.

60. The pharmaceutical composition according to claim 3, wherein the pellets are further mixed with at least one pharmaceutically acceptable excipient selected from one or more of filler, binder, glidant, disintegrant and lubricant before being compressed into tablets (or filled into capsules or sachets).

61. The pharmaceutical composition according to claim 60, wherein the filler is selected from the group consisting of microcrystalline cellulose, lactose monohydrate, maize starch, a composition comprising a spray dried mixture of lactose monohydrate and starch, powdered cellulose, sorbitol and mannitol.

62. The pharmaceutical composition according to claim 61, wherein the mixture of lactose monohydrate and starch is a composition of 85% alpha-lactose monohydrate and 15% maize starch by weight.

63. The pharmaceutical composition according to claim 60, wherein the binder is selected from the group consisting of povidone, hydroxypropyl cellulose, and hydroxypropyl methyl cellulose.

64. The pharmaceutical composition according to claim 60, wherein the glidant is selected from the group consisting of talc and silicon dioxide.

65. The pharmaceutical composition according to claim 60, wherein the disintegrant is selected from the group consisting of croscarmellose sodium, pregelatinized starch, crospovidone, hydroxypropyl cellulose, and sodium starch glycolate.

66. The pharmaceutical composition according to claim 60, wherein the lubricant is selected from the group consisting of stearic acid, magnesium stearate, mineral oil, hydrogenated castor oil and sodium stearyl fumarate.

67. The pharmaceutical composition according to claim 3, wherein the pellets are further mixed with a mixture of a filler, glidant, disintegrant and lubricant.

68. The pharmaceutical composition according to claim 67, wherein the pellets are mixed with a mixture of lactose monohydrate, colloidal silicon dioxide, sodium starch glycolate and magnesium stearate.

69. The pharmaceutical composition according to claim 68, wherein the lactose monohydrate is in the form of a composition comprising a spray dried mixture of lactose monohydrate and starch.

70. The pharmaceutical composition according to claim 69, wherein, the mixture is 85% alpha-lactose monohydrate and 15% maize starch by weight.

71. A process for preparing a pharmaceutical composition as defined in claim 1 comprising:
(a) forming pellets comprising zolpidem or a pharmaceutically acceptable salt thereof with at least one pharmaceutically acceptable excipient; and
(b) applying an extended release layer onto the pellets.

72. The process according to claim 71, wherein the pellets are further coated with a top coat layer.

73. The process according to claim 72, wherein the top coat layer is selected from: a mixture of hypromellose with at least one of titanium dioxide, polyethylene glycol and talc.

74. The process according to claim 71, wherein step (a) comprises (i) wet granulation of a mixture comprising zolpidem or a pharmaceutically acceptable salt thereof, a pellet former, a diluent and optionally a pH modifier, by extrusion spheronization to form cores, (ii) drying the cores, and optionally (iii) screening the cores.

75. The process according to claim 71, wherein step (b) comprises coating the cores with an extended release layer containing at least one release modifier and at least one plasticizer.

76. The process according to claim 71, wherein the pellets are further mixed with a mixture of a filler, glidant, disintegrant and lubricant.

77. The process according to claim 71, wherein the pellets are further mixed with a mixture of lactose monohydrate, colloidal silicon dioxide, sodium starch glycolate and magnesium stearate.

78. The process according to claim 77, wherein the lactose monohydrate is in a composition comprising a spray dried mixture of lactose monohydrate and starch.

79. The process according to claim 78, wherein the mixture is a composition of 85% alpha-lactose monohydrate and 15% maize starch by weight.

80. The process according to claim 71 further comprising mixing the plurality of pellets with at least one pharmaceutically acceptable excipient and compressing the mixture into tablets or filling the pellets into capsules or sachets.

81. A process for preparing the pharmaceutical compositions of claim 3 comprising:
(a) forming pellets comprising zolpidem or a pharmaceutically acceptable salt thereof with at least one pharmaceutically acceptable excipient;
(b) dividing the pellets into two populations;
(c) applying an extended release layer onto one of the populations of pellets; and
(d) applying a top coat layer.

82. The process according to claim 81, wherein step (a) comprises (i) wet granulation of a mixture comprising zolpidem or a pharmaceutically acceptable salt thereof, a pellet former, a diluent and optionally a pH modifier, by extrusion spheronization to form cores, (ii) drying the cores, and optionally (iii) screening the cores.

83. The process according to claim 81, wherein step (c) comprises coating the cores with an extended release layer containing at least one release modifier and at least one plasticizer.

84. The process according to claim 81, wherein step (d) comprises coating the pellets of both populations with a coating selected from: a mixture of hypromellose with at least one of titanium dioxide, polyethylene glycol and talc.

85. The process according to claim 81, wherein the pellets of both populations are mixed with at least one pharmaceutically acceptable excipient selected from one or more of a filler, binder, glidant, disintegrant and lubricant.

86. The process according to claim 85, wherein the filler is selected from the group consisting of microcrystalline cellulose, lactose monohydrate, maize starch, a composition comprising a spray dried mixture of lactose monohydrate and starch, powdered cellulose, sorbitol and mannitol; the binder is selected from the group consisting of povidone, hydroxypropyl cellulose, and hydroxypropyl methyl cellulose; the glidant is selected from the group consisting of talc and silicon dioxide; the disintegrant is selected from the group consisting of croscarmellose sodium, pregelatinized starch, crospovidone, hydroxypropyl cellulose, and sodium starch glycolate; and the lubricant is selected from the group consisting of stearic acid, magnesium stearate, mineral oil, hydrogenated castor oil and sodium stearyl fumarate.

87. The process according to claim 86, wherein the mixture of lactose monohydrate and starch is a composition of 85% alpha-lactose monohydrate and 15% maize starch by weight.

88. The process according to claim 85, wherein the pellets are further mixed with a mixture of a filler, glidant, disintegrant and lubricant.

89. The process according to claim 85, wherein the pellets are further mixed with a mixture of lactose monohydrate, colloidal silicon dioxide, sodium starch glycolate and magnesium stearate.

90. The process according to claim 89, wherein the lactose monohydrate is in a composition comprising a spray dried mixture of lactose monohydrate and starch.

91. The process according to claim 90, wherein the composition is 85% alpha-lactose monohydrate and 15% maize starch by weight.

92. The process according to claim 81, further comprising admixing the two populations of pellets with at least one pharmaceutical acceptable excipient and compressing the admixture into tablets or filling the pellets into capsules or sachets.

93. A pharmaceutical composition obtainable by the process of claim 71.

94. A pharmaceutical composition obtainable by the process of claim 81.
